(19) United States
(12) Patent Application Publication
(10) Pub. No.: US 2006/0079464 A1
(43) Pub. Date: Apr. 13, 2006

(54) Peritoneal dialysate containing taurine

(76) Inventors: Tsutomu Sanaka, Tokyo (JP); Maki Wakabayashi, Shizuoka (JP); Yukihiro Sano, Shizuoka (JP)

Correspondence Address: OSTRAGER CHONG FLAHERTY & BROITMAN PC, 250 PARK AVENUE, SUITE 825, NEW YORK, NY, 10177, US

(21) Appl. No.: 10/520,243

(22) PCT Filed: May 23, 2003
(86) PCT No.: PCT/JP03/06453

§ 371 Date: May 2, 2005

(30) Foreign Application Priority Data
Jul. 1, 2002 (JP) 2202-192177

(51) Int. Cl. A61K 31/70; A61K 31/185

(52) U.S. Cl. 514/23; 514/553

(57) ABSTRACT

[Purpose] It is an objective of the present invention to provide a stable neutral peritoneal dialysate that contains a substance other than glucose to serve as an osmotic agent. [Means to solve the problem] The peritoneal dialysate of the present invention is stable and can be provided in the form of a single solution in one-compartment containers.

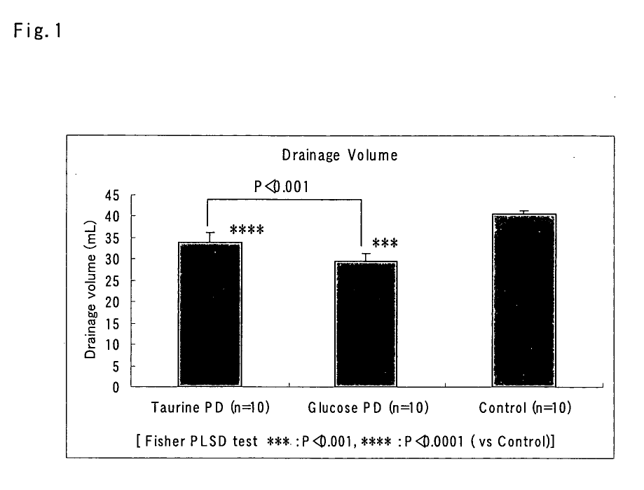

### DISCLOSURE OF THE INVENTION

1. Technical Field

The present invention relates to a perfusate preparation for use in peritoneal dialysis, including peritoneal dialysates for use in continuous ambulatory peritoneal dialysis (CAPD).

2. Background Art

Peritoneal dialysate such as a perfusate designed for use in CAPD, is designed to help patients suffering end stage renal failure to dispose of waste products through the peritoneum and thereby maintain the normal balance of various components of the bodily fluid. A typical CAPD perfusate contains electrolytes such as sodium chloride, calcium chloride, and magnesium chloride, as well as a lactate or a bicarbonate to serve as an alkalizer. It also contains glucose to serve as an osmotic agent that acts to keep the perfusate hypertonic, so that ultrafiltration of the perfusate is ensured.

Glucose has long been used as an osmotic agent in perfusates to establish a desired osmotic pressure because it is readily metabolized in the body, is effective in removing water, and is inexpensive. However, the potential effects that a high glucose level has on the body and its metabolism are now an issue of significant concern. For example, the peritoneum of a patient undergoing peritoneal dialysis is constantly exposed to the solution with high glucose concentration. Thus, the peritoneum will eventually deteriorate over the course of long-term dialysis and gradually lose its ability to remove water. In some cases, termination of the treatment is the only choice. In addition, a significant amount of glucose passes through the peritoneum into blood, increasing the blood glucose level. This not only makes the controlling of blood glucose level difficult in patients of diabetic nephropathy but also often leads to hyperinsulinemia in those who are non-diabetic. The high blood glucose level may also accompany hyperlipidemia.

Although it is desired that peritoneal dialysates have a neutral pH above 6.0 in the proximity of physiological pH, glucose tends to decompose in neutral or weakly basic pH ranges during the production and storage of the dialysates. As a result, the pH of glucose decreases over time, causing coloring of the solution or an increase in the amount of degraded products, such as 5-hydroxymethyl furfural (5-HMF), formic acid, and aldehydes. Not only are these degraded products cytotoxic, but also some reports suggest that they also facilitate the formation of advanced glycosylation end-products (AGE), compounds suspected to be involved in the development of amyloidosis or other complications. For this reason, peritoneal dialysates are typically designed to show a slightly acidic pH. This, however, irritates the peritoneum and facilitates its deterioration.

To counteract these problems, one technique uses a container having two separate compartments so that glucose can be stored separately from the components that facilitate the decomposition of glucose, while another approach provides a glucose solution in small volumes but at a high concentration (See, for example, Japanese Patent Laid-Open Publication No. Hei 3-195561, Japanese Patent Laid-Open Publication No. 2000-51348, Japanese National Publication No. Hei 7-500992, and International Patent Publication No. WO99/09953). The approach to use the two-compartment container, however, is rather complicated since it requires mixing of the two formulations by removing a separator or opening a clip. Also, the technique still involves the use of a solution with high glucose concentration, and the problem of the effects of high glucose concentration on the body and its metabolism is left unattended.

Also, much effort has been devoted to finding an alternative to glucose that can serve as an ideal osmotic agent. Among the potential alternatives that have been proposed thus far are amino acids and polypeptides, which are described in Japanese Patent No.3065352 and Japanese Patent Publication No. Hei 7-504351, respectively. One drawback of these approaches is that the blood urea nitrogen (BUN) levels tend to rise. Also, in some cases, only a less volume of water was removed in these approaches than is possible by the use of glucose. Another type of peritoneal dialysate disclosed in Japanese Patent Nos. 1824784, 2120679, and 2106222 makes use of glucose polymers or the like. Though in small amounts, the absorption of these polymers by the living body and the accumulation of the polymers and the degraded products in the body pose a significant problem.

### THE PROBLEM TO BE SOLVED IN THE INVENTION

Accordingly, it is an objective of the present invention to eliminate the problems of the conventional techniques by providing a stable neutral peritoneal dialysate that contains a substance other than glucose to serve as an osmotic agent.

### MEANS TO SOLVE THE PROBLEM

In an effort to find a solution to the aforementioned problems, the present inventors have made a finding that, by using a taurine compound as an alternative to glucose to serve as an osmotic agent, a stable neutral peritoneal dialysate can be provided. This finding ultimately led the present inventors to complete the present invention. As used herein, the term “taurine compound” includes, aside from taurine itself, any precursor of taurine, such as hypotaurine and thiotaurine.

Taurine, also known as 2-aminoethanesulfonic acid, acts as an osmotic agent that helps cells maintain a desired osmotic balance against hypertonic extracellular conditions created by urea and electrolytes during the urine concentration in kidneys. Taurine is abundant in the body and is synthesized in vivo from methionine via cysteine. There have been some reports suggesting that the synthesis of taurine is inhibited in patients undergoing CAPD and taurine levels in plasma and muscles in these patients remain low.

This implies that a solution containing a taurine compound can serve as an effective peritoneal dialysate that has minimum effects on the body and its metabolism.

Accordingly the present invention provides:

(1) a peritoneal dialysate containing a taurine compound along with an electrolyte and an alkalizer;

(2) The peritoneal dialysate according to (1), wherein the alkalizer is a lactate, a citrate, or a bicarbonate, and the electrolyte is sodium ion, calcium ion, magnesium ion, or chloride ion;

(3) The peritoneal dialysate according to claims 1 or 2, having an osmotic pressure of 300 to 680 mOsm;

(4) The peritoneal dialysate according to any one of claims 1 to 3, wherein the pH upon use is adjusted to a value of 6.0 to 7.5;

(5) The peritoneal dialysate according to any one of claims 1 to 4, provided in a one-compartment container;

(6) The peritoneal dialysate according to any one of claims 1 to 4, provided in a one-compartment container; and

(7) A peritoneal dialysate, containing 1 to 5 w/v % of taurine, 25 to 45 mEq/L of sodium lactate, 110 to 150 mEq/L of sodium ion, 0.5 to 5 mEq/L of calcium ion, 0.1 to 2.0 mEq/L of magnesium ion, and 80 to 110 mEq/L of chloride ion and having a pH of 6.0 to 7.5.

### BEST MODE FOR IMPLEMENTING THE INVENTION

One characteristic of the present invention resides in that a taurine compound is added to a peritoneal dialysate to serve as an osmotic agent. Taurine, an amphoteric ion, exhibit a neutral pH when dissolved in water and has the ability to buffer pH changes. For this reason, taurine can be used to stabilize the pH of peritoneal dialysates during sterilization and storage. Furthermore, taurine is more stable against the sterilization process than glucose, which is advantageous since, through the use of taurine, a neutral peritoneal dialysate can be formulated as a single solution that can be stored in a single compartment container. The present invention also takes advantage of physiological activities of taurine for the purposes of improving functions of livers and circulatory systems, improving lipid metabolism, and facilitating diuresis.

Preferably, the amount of taurine compound to serve as an osmotic agent is from 1 to 5 w/v %. The peritoneal dialysate is preferably adjusted to have an osmotic pressure of 300 to 680 mOsm and more preferably 300 to 500 mOsm, while the osmotic pressure may vary depending on the amount of ions of electrolytes in the peritoneal dialysate.

According to the present invention, a taurine compound may be added to the peritoneal dialysate along with glucose. When taurine is present in the dialysate together with glucose, the ability of taurine to serve as a buffer helps maintain the pH of the dialysate at a neutral value. To this end, the amount of taurine is preferably from 0.01 to 5 w/v %.

An alkalizer for use in the present invention may be a lactate, a citrate, or a hydrogencarbonate. An electrolyte for use in the present invention includes sodium ion, calcium ion, magnesium ion, or chloride ion, each of which is commonly in use in peritoneal dialysis. The electrolytes are preferably used in the form of sodium chloride, calcium chloride, and magnesium chloride. Aside from the components above, the peritoneal dialysate of the present invention may further contain various amino acids, trace elements, and other components commonly in use in peritoneal dialysates.

The peritoneal dialysate of the present invention preferably contains each of the above-described components in the following concentration ranges:

| | | |
|---|---|---|
| sodium ion | 110 to 150 | mEq/L |
| calcium ion | 0.5 to 5 | mEq/L |
| magnesium ion | 0.1 to 2.0 | mEq/L |
| chloride ion | 80 to 110 | mEq/L |
| alkalizer | 25 to 45 | mEq/L |
| glucose | 0 to 4 | w/v % |
| taurine compound | 0.01 to 5 | w/v % |

Preferably, the peritoneal dialysate has a pH of 6.0 to 7.5. A pH conditioner for use in the peritoneal dialysate may be any commonly used pH conditioner, including sodium hydroxide, sodium hydrogencarbonate, hydrochloric acid, lactic acid, and citric acid.

According to the present invention, the addition of taurine compound as an alternative to glucose to serve as an osmotic agent permits formulation of a stable peritoneal dialysate as a single solution, although, if necessary, the dialysate may be provided in the form of two or more separate solutions.

The peritoneal dialysate is generally provided in a plastic container made of such materials as polyethylene, polypropylene, polyvinyl chloride, polyester, ethylene/vinyl acetate copolymer, nylon, or composite materials thereof. This container preferably includes a single compartment for holding the dialysate while it may include two or more compartments if desired.

While the peritoneal dialysate can be sterilized by common heating process, it may also be sterilized in a proper manner by a sterile filtration process.

When necessary, containers of the peritoneal dialysate may be packaged by gas barrier material or the dialysate may be placed in plastic containers having the same property. The gas barrier property is a property of a material that permits little or no penetration of gases such as oxygen, nitrogen, carbon dioxide, and water vapor. Examples of the plastic material having the gas barrier property includes ethylene/vinyl alcohol copolymer, polyvinylidene chloride, nylon with gas barrier property, plastic materials coated or laminated with these resins, or plastic materials coated with a thin film of aluminum, aluminum oxide, silicon oxide or other proper materials. This plastic material may or may not be transparent.

When it is desired to package the container of the peritoneal dialysate by the gas barrier material, the space between the container and the material may be filled with gaseous nitrogen, carbon dioxide or other inert gases, which may be used independently or as a proper mixture. Alternatively, the container of the peritoneal dialysate may be packaged with the gas barrier material while air is removed.

### EXAMPLES

The present invention will now be described with reference to Examples.

### Example 1

107.6 g of sodium chloride, 5.14 g of calcium chloride dehydrate, 1.016 g of magnesium chloride hexahydrate, 179.2 g of 50% sodium lactate solution, and 200 g of taurine were dissolved in a proper amount of water for injection. Sodium hydroxide was then added to adjust the pH of the solution to 7 and to give a final volume of 20L. This solution was designated as a Test Solution 1. Similarly, three solutions, having the same composition as Test Solution 1 but containing 360 g and 560 g of taurine, respectively, were prepared and were designated as Test Solutions 2 and 3, respectively. A solution containing 300 g of glucose in place of taurine was designated as a Comparative Solution. 1500 mL of each solution was placed in a polypropylene bag and was sterilized in an autoclave.

Each solution was observed before and after the sterilization and after being stored for 2 weeks at 60° C. at 30% RH and was examined for any changes. The results are shown in Table 1 below.

TABLE 1

| | Examined | Before | After | 2 weeks |
|---|---|---|---|---|
| Sample | properties | sterilization | sterilization | later |
| Test Sltn 1 | Appearance | Clear and | Clear and | Clear and |
| | | colorless | colorless | colorless |
| | pH | 7.34 | 7.36 | 7.34 |
| | O.P.(mOsm) | 324 | 325 | 324 |
| | Taurine (w/v %) | 0.97 | 0.97 | 0.96 |
| Test Sltn 2 | Appearance | Clear and | Clear and | Clear and |
| | | colorless | colorless | colorless |
| | pH | 7.30 | 7.31 | 7.30 |
| | O.P.(mOsm) | 386 | 386 | 385 |
| | Taurine (w/v %) | 1.76 | 1.79 | 1.79 |
| Test Sltn 3 | Appearance | Clear and | Clear and | Clear and |
| | | colorless | colorless | colorless |
| | pH | 7.29 | 7.29 | 7.29 |
| | O.P.(mOsm) | 466 | 467 | 466 |
| | Taurine (w/v %) | 2.76 | 2.76 | 2.75 |
| Comp. Sltn | Appearance | Clear and | Clear and | Clear and |
| | | colorless | colorless | faint |
| | | | | yellow |
| | pH | 7.13 | 6.18 | 5.85 |
| | O.P.(mOsm) | 337 | 338 | 338 |
| | Glucose (w/v %) | 1.49 | 1.41 | 1.41 |

O.P. = osmotic pressure

As shown in Table 1 above, no significant change was observed in the appearance, pH, osmotic pressure, or the taurine content in any of Test Solutions 1, 2 and 3 after autoclaving and after the 2-week storage period at 60° C., proving the stability of each Test Solution. In comparison, the pH of Comparative Solution was significantly decreased after autoclaving, as was its glucose content. After the 2-week storage period, Comparative Solution was colored and its pH was significantly changed. This indicates that Comparative Solution is unstable.

### Example 2

10.1 g of taurine, 179.2 g of 50% sodium lactate solution, 107.6 g of sodium chloride, and 1.02 g of magnesium chloride hexahydrate were dissolved in 10L of water for injection, followed by the addition of sodium hydroxide to adjust the pH of the solution to 7.6. Meanwhile, 272 g of glucose and 5.14 g of calcium chloride dehydrate were dissolved in 10L of water for injection. Hydrochloric acid was then added to adjust the pH of the solution to 4.2. 750 mL of each solution was placed in each compartment of a two-compartment polypropylene bag. After the bag was autoclaved, the solutions were mixed with each other to form Test Solution 4. Similarly, two solutions, having the same composition as Test Solution 4 but containing 15.2 g and 20.2 g of taurine, respectively, were prepared and were designated as Test Solutions 5 and 6, respectively. Also, a taurine-free solution was prepared to serve as a Comparative Solution. Each solution was observed after the mixing and after being stored for 10 days at 40° C. at 75% RH and was examined for any changes. The results are shown in Table 2 below.

TABLE 2

| | Examined | | |
|---|---|---|---|
| Sample | properties | After mixing | 10 days later |
| Test Sltn 4 | Appearance | Clear and | Clear and |
| | | colorless | colorless |
| | PH | 7.33 | 7.14 |
| Test Sltn 5 | Appearance | Clear and | Clear and |
| | | colorless | colorless |
| | PH | 7.40 | 7.19 |
| Test Sltn 6 | Appearance | Clear and | Clear and |
| | | colorless | colorless |
| | PH | 7.40 | 7.22 |
| Comp. Sltn | Appearance | Clear and | Clear and |
| | | colorless | colorless |
| | PH | 7.27 | 6.83 |

As can be seen from the results, no significant change was observed in the appearance and pH in any of Test Solutions 4, 5, and 6 as compared to Comparative Solution, indicating the stability of each Test Solution.

### Example 3

Comparative Solution G1 was prepared that contained 5.38 g of sodium chloride, 0.257 g of calcium chloride dihydrate, 0.0508 g of magnesium chloride hexahydrate, 8.96 g of 50% sodium lactate solution, and 13.6 g of glucose per 1L. The solution was sterilized in a two-compartment container and was adjusted so that the pH of the solution upon use would be 7. Similarly, two solutions, having the same composition as Comparative Solution G1 but containing 22.7 g and 38.6 g of glucose, respectively, were prepared and were designated as Comparative Solutions G2 and G3, respectively. 30 mL of each solution was injected into the abdominal cavity of male SD rats. After 4 hours, the volume of abdominal fluid was measured and the difference between the volumes of the abdominal fluid and the administered solution was taken to give the volume of removed water. The results are shown in Table 3 below.

TABLE 3

| | | | | |
|---|---|---|---|---|
| | | Average | Minimum | Maximum |
| | Conc. of | volume of | volume of | volume of |
| | taurine or | removed | removed | removed |
| | glucose | water | water | water |
| Sample | (w/v %) | (mL) | (mL) | (mL) |

Taurine-containing test solutions

| | | | | |
|---|---|---|---|---|
| Test Sltn T1 | 1.0 | 0.2 | −1.4 | 1.2 |
| Test Sltn T2 | 1.8 | 8.2 | 6.6 | 9.5 |
| Test Sltn T3 | 2.8 | 13.3 | 12.3 | 14.5 |

Glucose-containing controls

| | | | | |
|---|---|---|---|---|
| Comp. Sltn G1 | 1.36 | 3.9 | 1.2 | 6.0 |
| Comp. Sltn G2 | 2.27 | 11.2 | 10.3 | 13.0 |
| Comp. Sltn G3 | 3.86 | 18.2 | 16.5 | 21.3 |

These results indicate that, through the use of taurine, water was removed in a concentration-dependent manner as in the case of the conventional glucose formulation.

### EFFECT OF THE PRESENT INVENTION

As set forth, the neutral peritoneal dialysate of the present invention, which contains a taurine compound as an osmotic agent, does not bring about the problem of coloring of the dialysate due to decomposition of glucose or the problem of degraded products of glucose. Also, the peritoneal dialysate of the present invention is stable and can be provided in the form of a single solution in one-compartment containers. Because the taurine-containing peritoneal dialysate of the present invention exhibits a good biocompatibility, blood suger level can be controlled on diabetic patient and it dose not cause the degeneration of the peritoneum mesothelial cell.

1. A peritoneal dialysate containing a taurine compound along with an electrolyte and an alkalizer.

2. The peritoneal dialysate according to claim 1, wherein the alkalizer is a lactate, a citrate, or a bicarbonate, and the electrolyte is sodium ion, calcium ion, magnesium ion, or chloride ion.

3. The peritoneal dialysate according to claims 1 or 2, having an osmotic pressure of 300 to 680 mOsm.

4. The peritoneal dialysate according to any one of claims 1 to 3, wherein the pH upon use is adjusted to a value of 6.0 to 7.5.

5. The peritoneal dialysate according to any one of claims 1 to 4, provided in a one-compartment container.

6. A peritoneal dialysate, containing 0.01 to 5 w/v % of taurine, 25 to 45 mEq/L of sodium lactate, 110 to 150 mEq/L of sodium ion, 0.5 to 5 mEq/L of calcium ion, 0.1 to 2.0 mEq/L of magnesium ion, 80 to 110 mEq/L of chloride ion, and 0 to 4 w/v % of glucose and having a pH of 6.0 to 7.5 upon use.

7. A peritoneal dialysate, containing 1 to 5 w/v % of taurine, 25 to 45 mEq/L of sodium lactate, 110 to 150 mEq/L of sodium ion, 0.5 to 5 mEq/L of calcium ion, 0.1 to 2.0 mEq/L of magnesium ion, and 80 to 110 mEq/L of chloride ion and having a pH of 6.0 to 7.5.
